(19) United States
(12) Patent Application Publication
(10) Pub. No.: US 2010/0016781 A1
(43) Pub. Date: Jan. 21, 2010

(54) Iontophoresis device selecting drug to be administered on the basis of information form sensor

(76) Inventors: Mizuo Nakayama, Shibuya-ku (JP); Takehiko Matsumura, Shibuya-ku (JP); Hidero Akiyama, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/991,234

(22) PCT Filed: Aug. 29, 2006
(86) PCT No.: PCT/JP2006/316933

§ 371 Date: Aug. 31, 2009

(30) Foreign Application Priority Data
Aug. 29, 2005 (JP) 2005-247986

(51) Int. Cl. A61N 1/30

(52) U.S. Cl. 604/20

(57) ABSTRACT

An iontophoresis device including an electric power source. A first electrode assembly is electrically coupled to the electric power source to transdermally administer an ionic drug to an organism via iontophoresis while a second electrode assembly is electrically coupled to the electric power source as a counter electrode to the first electrode assembly. A sensor may be positioned proximate an internal or external portion of the organism and operable to determine information used to select and deliver the ionic drug to the organism.

### BACKGROUND

1. Field

The present disclosure relates to a technique of transdermally administering various ionic drugs (transdermal drug delivery) by iontophoresis. In particular, the present disclosure relates to an iontophoresis device adapted to select a drug to be administered on the basis of information from a sensor set in an organism and to release the drug selected on the basis of the selection result.

2. Description of the Related Art

A method of introducing (permeating) an ionic drug placed on the surface of the skin or mucosa (hereinafter, merely referred to as “skin”) of a predetermined site of an organism into the body through the skin by giving the skin an electromotive force sufficient to drive such ionic drug is called iontophoresis (iontophorese, ion introduction method, ion permeation therapy) (See e.g., JP 63-35266 A).

For example, positively charged ions are driven (transported) into the skin on the side of an anode (positive electrode) in an electric system of an iontophoresis device. On the other hand, negatively charged ions are driven (transported) into the skin on the side of a cathode (negative electrode) in the electric system of the iontophoresis device.

Conventionally, a large number of such iontophoresis devices as described above have been proposed (see, for example, JP 63-35266 A, JP 04-297277 A, JP 2000-229128A, JP 2000-229129A, JP 2000-237327A, JP 2000-237328 A, and WO 03/037425 A1).

A drug to be administered to an organism is selected in such conventional iontophoresis device as described above generally in accordance with a prior instruction by a prescription or the judgment of a person who applies a drug or the like.

However, when drugs to be administered cover a broad spectrum in accordance with symptoms, or when a variety of drugs must be quickly and accurately administered to a large number of patients, the selection of a drug to be administered only by a human being is limited.

### BRIEF SUMMARY

In some embodiments an iontophoresis device may enable a drug to be administered to be quickly and accurately selected and administered.

According to another embodiment, an iontophoresis device includes, an electric power source, a first electrode assembly electrically coupled to the electric power source to transdermally administer an ionic drug to an organism via iontophoresis, a second electrode assembly electrically coupled to the electric power source as a counter electrode to the first electrode assembly, and a sensor positioned proximate an internal or external portion of the organism and operable to determine information used to select and deliver the ionic drug to the organism.

In another embodiment, the first electrode assembly includes, a first electrode electrically coupled to the electric power source to have a same polarity as a component of the ionic drug, a first electrolyte solution holding portion impregnated with a first electrolyte solution, the first electrolyte solution holding portion disposed adjacent to the first electrode, a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, a drug holding portion impregnated with the ionic drug, the drug holding portion disposed adjacent to the first ion exchange membrane, and a second ion exchange membrane that substantially passes ions having a polarity opposite the polarity of the ionic drug and that substantially blocks ions having a polarity that is the same as a polarity of the ionic drug, the ion exchange membrane disposed adjacent to the drug holding portion.

According to yet another embodiment, the second electrode assembly includes, a second electrode electrically coupled to the electric power source to have a polarity opposite that of the first electrode, a second electrolyte solution holding portion impregnated with a second electrolyte solution, the second electrolyte solution holding portion disposed adjacent to the second electrode, and a third ion exchange membrane that substantially passes ions having a polarity that is the same as a polarity of the ionic drug and that substantially blocks ions having a polarity that is opposite the polarity of the ionic drug, the ion exchange membrane disposed adjacent to the second electrolyte solution holding portion.

In a further embodiment of the iontophoresis device, the first electrode assembly may include a plurality of electrode assemblies capable of independently releasing a plurality of types of selected drugs.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a schematic illustration of an iontophoresis device, according to one illustrated embodiment.

FIG. 2A is a block diagram depicting a process for selecting and delivering an ionic drug by way of the iontophoresis device, according to one illustrated embodiment.

### DETAILED DESCRIPTION

As described above, the iontophoresis device according to one embodiment includes a system for selecting and administering an appropriate drug to be administered on the basis of detection information from a sensor set in an organism that is an object to be administered with the drug, the system being characterized in that the drug to be administered is selected on the basis of information from the sensor. The sensor may be set in the organism to be administered with the drug, and the selected drug may be released on the basis of a selection result. Therefore, an appropriate drug can be administered quickly in accordance with the condition of an organism.

As described above, the iontophoresis device according to one embodiment includes: an electric power source device; a first electrode assembly for administering an ionic drug to an organism transdermally by releasing the ionic drug by iontophoresis, the first electrode assembly being connected to the electric power source device; and a second electrode assembly as a counter electrode of the first electrode assembly, and is characterized in that a drug to be administered is selected on the basis of information from a sensor. The sensor may be set in the organism to be administered with the drug, and the selected drug may be released on the basis of a selection result.

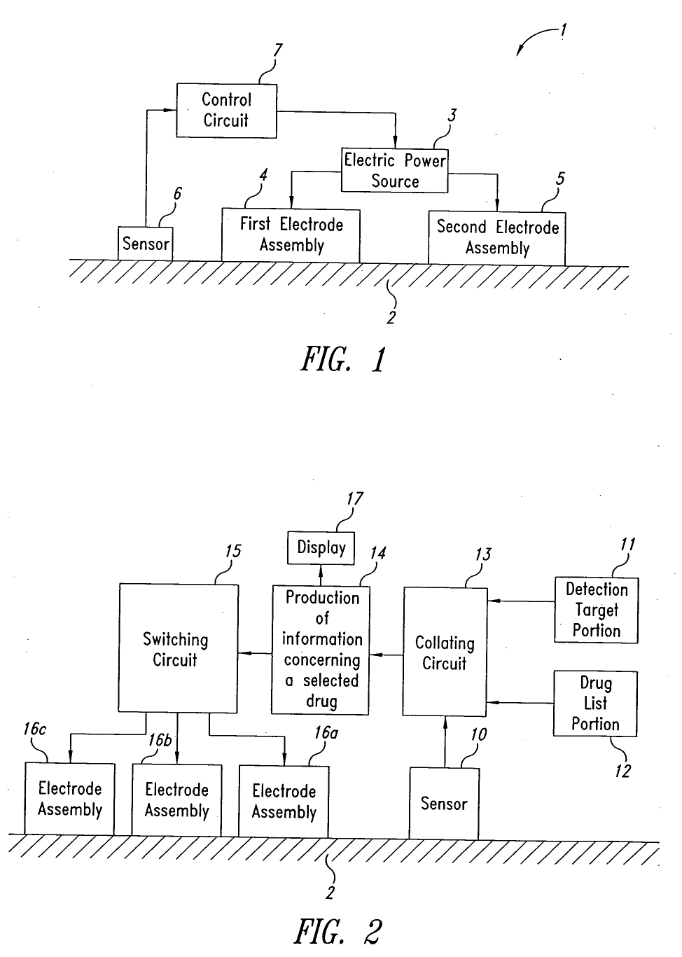

Hereinafter, some embodiments will be described on the basis of specific examples shown in the drawings. FIG. 1 shows an iontophoresis device 1 according to one illustrated embodiment. The iontophoresis device 1 may be placed on the surface of an organism 2 (e.g. skin or mucosa). The device 1 may include an electric power source 3, a first electrode assembly 4 and a second electrode assembly 5. The first and second electrode assemblies 4, 5 being connected to the electric power source 3. Furthermore, the iontophoresis device 1 includes a sensor 6 which may be set in a specific site of the organism 2. Information from the sensor 6 may be subjected to processing in a control circuit 7. The processed information may be fed back to the electric power source 3 to thereby control an operation in the first electrode assembly 4 or second electrode assembly 5.

For example, the sensor 6 may be independent or incorporated into a catheter or the like and placed in a blood vessel of the organism 2. Description will be given of the case where the sensor 6 is a blood glucose level sensor (e.g., glucosensor) and the drug to be administered is insulin. The blood glucose level sensor is a sensor capable of measuring a blood glucose level in the blood for a short time period, and measure the blood glucose level with or without a substantially painless fine needle. The measured data may be sent to the control circuit 7 to be subjected to data processing and used as feedback information in determining an ON/OFF operating status of the electric power source3. The ON/OFF operating status of the release of, for example, insulin from the first electrode assembly 4 is controlled in accordance with the processed data.

Alternatively or additionally, the sensor 6 may include, for example, a blood pressure sensor. The drug to be administered in accordance with information from the blood pressure sensor may, for example, be a hypotensive drug. Information pertaining to a blood pressure value may be sent from the sensor 6 to the control circuit 7. is the information may be subjected to a control processing when the blood pressure value reaches a preset blood pressure value to determine the ON/OFF operating status of the first electrode assembly 2 for releasing the hypotensive drug. The ON/OFF operating status of the release of the hypotensive drug from the first electrode assembly 2 is controlled in accordance with the processed information.

FIG. 2 shows a block diagram illustrating the process described above in which a drug to be administered is selected on the basis of information from a sensor and the drug is administered on the basis of a selection result.

The iontophoresis device, as shown in FIG. 2 includes a detection target portion 11 and a drug list portion 12. The detection target portion 11 may provide information about a detection target such as a detection target component in the blood (e.g., a blood glucose level or a blood pressure value). The drug list portion 12 may provide information of types of drugs (e.g., insulin or a hypotensive drug) to be administered. The information of the types of drugs may, for example, be organized in the form of a list. A collating circuit 13 collates the information provided from both the detection target portion 11 and the drug list portion 12 together with information from a sensor 10. Information concerning a selected drug to be administered is produced in a circuit 14 on the basis of the result of the collation. The produced information concerning the selected drug may be displayed on, for example, a display device 17, and a person (e.g., medical practitioner) who applies a drug can quickly perform an operation necessary for the administration of the selected drug on the basis of the displayed information.

Alternatively, the selected drug may be automatically administered. For example, a plurality of electrode assemblies 16a, 16b, 16c operable to release respective drugs stored therein may be switched by a switching circuit 15 communicatively coupled to the electrode assemblies 16a, 16b, 16c. The switching circuit 15 may switch between respective ones of the plurality of assemblies 16a, 16b, 16c on the basis of, for example, information from the circuit 14 pertaining to information of the selected drug. Once the switching circuit 15 selects one of the plurality of assemblies 16a, 16b, 16c, the drug may be released.

The sensor 10 to detect and measure useful information from the organism 2 in accordance with the drug to be administered is not limited to the blood glucose level sensor or blood pressure gauge as described above. A large number of sensors related to drugs that can be administered by iontophoresis are applicable. Specifically, in addition to the blood pressure gauge for measuring a blood pressure or the blood glucose level sensor as described above, a sensor for detecting a specific target component in the blood, for example, a heart rate meter for measuring a heart rate, a pulse rate meter for measuring a pulse rate, a clinical thermometer, a moisture meter, an inspirometer, a blood flow meter, and the like are independently or compositely applicable.

According to one embodiment of the iontophoresis device 1, the first electrode assembly 4 may include, a first electrode electrically coupled to the electric power source to have a same polarity as a component of an ionic drug, a first electrolyte solution holding portion impregnated with a first electrolyte solution, the first electrolyte solution holding portion disposed adjacent to the first electrode plate, a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, a drug holding portion impregnated with the ionic drug, the drug holding portion disposed adjacent to the first ion exchange membrane, and a second ion exchange membrane that substantially passes ions having a polarity opposite the polarity of the ionic drug and that substantially blocks ions having a polarity that is the same as a polarity of the ionic drug, the ion exchange membrane disposed adjacent to the drug holding portion. The application of WO 03/037425 A1 mentioned above describes details of an iontophoresis device including such electrode assembly.

According to one embodiment, in the iontophoresis device 1, the first electrode assembly 4 may be a working electrode assembly, and the second electrode assembly 5 may be a non-working electrode assembly (ground electrode assembly). However, in some embodiments, both the first and the second electrode assemblies 4, 5 may be operable to release drugs, and such embodiments are also included in embodiments of the present invention.

In addition, as described above, in some embodiments the first electrode assembly 4 may include a plurality of electrode assemblies capable of releasing a plurality of types of selected drugs independently.

In addition, an inactive electrode made of a conductive material such as carbon or platinum may be used as the electrode of the electrode assembly. The electrolyte solution holding portion can be constituted by a thin film that has the property of holding an electrolyte solution by being impregnated with the electrolyte solution. The thin film can be made of the same material as that used for a drug holding portion holding an ionic drug by being impregnated with the ionic drug to be described later.

A desired one can be appropriately used as the electrolyte solution depending upon the conditions such as a drug to be applied. However, an electrolyte solution that damages the skin of the organism 2 owing to an electrode reaction should be avoided. An organic acid or a salt thereof present in a metabolic cycle of the organism 2 may be preferable as the suitable electrolyte solution in one embodiment of the present invention in terms of harmlessness. For example, lactic acid and fumaric acid may be preferable. Specifically, an aqueous solution of 1M of lactic acid and 1M of sodium fumarate (1:1) may be preferable. Such electrolyte solution may be preferable because it has a high solubility with respect to water and passes a current well. In the case where a current is allowed to flow at a constant level, the electric resistance is low and a change in pH is relatively small in an electric power source device.

A cation exchange membrane and an anion exchange membrane may preferably be used together as ion exchange membranes to be used for an electrode assembly. Furthermore, the drug holding portion includes a thin film that holds an ionic drug by being impregnated with the ionic drug. Such thin film may have a sufficient ability to hold an ionic drug by being impregnated with the ionic drug, and a sufficient ability to move the ionic drug (e.g., ion transferability, ion conductivity), which is impregnated in and held by the thin film, into the skin side of the organism 2 under predetermined electric field conditions. The following conditions may, for example, typically be adopted as operating conditions in an iontophoresis device as described above.

(1) Constant current condition, specifically, 0.1 to 0.5 mA/cm², preferably 0.1 to 0.3 mA/cm²

(2) Safe voltage condition that realizes the above constant current, specifically, 50 V or less, preferably 30 V or less

Examples of a material that brings together good property of holding a drug by being impregnated with the drug and good ion transferability include hydrogel forms of acrylic resins (acrylic hydrogel film), a segmented polyurethane-based gel film, and an ion-conductive porous sheet for forming a gel-like solid electrolyte.

Specific examples of an ionic drug applicable to iontophoresis may include anesthetic drugs (e.g., procaine hydrochloride and lidocaine hydrochloride) as well as the above-described drugs such as, for example, insulin and a hypotensive drug. They are appropriately selected depending on uses.

International Patent Application No. WO 03/037425 A1 mentioned above, describes details about the above-described respective components and operating conditions, and the contents described in the document are also included in various embodiments of the present invention.

The various embodiments described above can be combined to provide further embodiments. All of the U.S. patents, U.S. patent application publications, and U.S. patent applications referred to in this specification, are incorporated herein by reference, in their entirety. Embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An iontophoresis device, comprising:
an electric power source;
a first electrode assembly electrically coupled to the electric power source to transdermally administer an ionic drug to an organism via iontophoresis;
a second electrode assembly electrically coupled to the electric power source as a counter electrode to the first electrode assembly; and
a sensor positioned proximate an internal or external portion of the organism and operable to determine information used to select and transdermally deliver the ionic drug to the organism.

2. The iontophoresis device according to claim 1 wherein the first electrode assembly comprises:
a first electrode electrically coupled to the electric power source to have a same polarity as a component of the ionic drug;
a first electrolyte solution holding portion impregnated with a first electrolyte solution, the first electrolyte solution holding portion disposed adjacent to the first electrode;
a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion;
a drug holding portion impregnated with the ionic drug, the drug holding portion disposed adjacent to the first ion exchange membrane; and
a second ion exchange membrane that substantially passes ions having a polarity opposite the polarity of the ionic drug and that substantially blocks ions having a polarity that is the same as a polarity of the ionic drug, the second ion exchange membrane disposed adjacent to the drug holding portion.

3. The iontophoresis device according to claim 2 wherein the second electrode assembly comprises:
a second electrode electrically coupled to the electric power source to have a polarity opposite that of the first electrode;
a second electrolyte solution holding portion impregnated with a second electrolyte solution, the second electrolyte solution holding portion disposed adjacent to the second electrode; and
a third ion exchange membrane that substantially passes ions having a polarity that is the same as a polarity of the ionic drug and that substantially blocks ions having a polarity that is opposite the polarity of the ionic drug, the ion exchange membrane disposed adjacent to the second electrolyte solution holding portion.

4. The iontophoresis device according to claim 1 wherein the first electrode assembly comprises a plurality of electrode assemblies capable of releasing respective ones of a plurality of ionic drugs.

5. The iontophoresis device according to claim 4 wherein the sensor is operable to determine the information used to select and transdermally deliver the respective ones of the plurality of ionic drugs to the organism.
